(19) United States
(12) Patent Application Publication
(10) Pub. No.: US 2026/0325770 A1
(43) Pub. Date: Oct. 22, 2026

(54) INHALER

(71) Applicant: KT&G CORPORATION, Daejeon (KR)

(72) Inventors: Min Kyu KIM, Guri-si (KR); Paul Joon SUNWO0O, Seoul (KR); Wonkyeong LEE, Seoul (KR)

(73) Assignee: KT&G CORPORATION, Daejeon (KR)

(21) Appl. No.: 19/108,643

(22) PCT Filed: Sep. 1, 2023
(86) PCT No.: PCT/KR2023/013076

§ 371 Date: Mar. 4, 2025

(30) Foreign Application Priority Data
Sep. 6, 2022 (KR) 10-2022-0112643
Nov. 17, 2022 (KR) 10-2022-0154394

(51) Int. Cl. A61M 15/06; A61M 15/00

(52) U.S. Cl. CPC A61M 15/06; A61M 15/0006; A61M 15/0035; A61M 15/0041; A61M 2202/064; A61M 2205/3334

(57) ABSTRACT

An inhaler according to an embodiment includes a stick including a chamber configured to accommodate powder, a holder including an insertion groove into which the stick is inserted, a fixing member fixed to the insertion groove, an impact member connected to the fixing member, and a rotating member including a rotation axis provided in the insertion groove and at least one rotation blade provided on an outer circumferential surface of the rotating member, wherein the rotating member is configured to press the impact member temporarily while the rotation blade and the impact member are engaged with each other, as the rotating member rotates around the rotation axis.

### TECHNICAL FIELD

Various embodiments of the present disclosure relate to an inhaler.

### BACKGROUND ART

Recently, demands for alternative articles to overcome disadvantages of general cigarettes have increased. For example, an inhaler is a device for inhaling a liquid or gas containing a composition such as a drug by a user through the oral cavity or nasal cavity of the user.

Such a device may have a chamber accommodating an inhalable composition, and the inhalable composition may pass from the chamber through a channel and finally to the oral or nasal cavity of a user to be inhaled by the user.

The above description has been possessed or acquired by the inventor(s) in the course of conceiving the present disclosure and is not necessarily an art publicly known before the present application is filed.

### DISCLOSURE OF THE INVENTION

### Technical Goals

In an inhaler using a composition in a powder state, a non-electronic inhaler according to the related art has to inhale the powder depending on the breathing of a user. In this case, the amount of powder discharged from the inhaler may vary according to the lung capacity of the user, and a user whose lung capacity does not meet a certain level has a problem in that the use of the inhaler is restricted.

To solve this problem, there is a demand for an inhaler capable of smoothly inhaling even by a user with weak lung capacity, and furthermore, individually controlling a discharged state of powder to a general user.

### Technical Solutions

According to an embodiment, an inhaler includes a stick including a chamber configured to accommodate powder, a holder including an insertion groove into which the stick is inserted, a fixing member fixed to the insertion groove, an impact member connected to the fixing member, and a rotating member including a rotation axis provided in the insertion groove and at least one rotation blade provided on an outer circumferential surface of the rotating member. The rotating member is configured to press the impact member temporarily while the rotation blade and the impact member are engaged with each other, as the rotating member rotates around the rotation axis.

In an embodiment, one end portion of the impact member may be a fixed end that is connected to the fixing member and the other end portion opposite to the one end portion of the impact member is a free end.

In an embodiment, the other end portion of the impact member may be tilted in a direction away from the chamber when the impact member is pressed by the rotation blade and the other end portion of the impact member may move toward the chamber and be configured to impact the chamber when the impact member is separated from the rotation blade.

In an embodiment, the other end portion of the impact member may bounce toward the chamber and be configured to impact the chamber when the impact member is pressed by the rotation blade.

In an embodiment, the impact member may be arranged to face one surface of the stick facing a direction into which the stick is inserted in the insertion groove.

In an embodiment, the impact member may be arranged to face a side surface adjacent to one surface of the stick facing a direction into which the stick is inserted in the insertion groove.

In an embodiment, the impact member and the rotating member may configure one impact module and the inhaler may include the impact module in a plurality.

In an embodiment, the impact modules may be arranged in opposite directions to each other based on the chamber.

In an embodiment, one impact member and another impact member of the impact modules may be configured to impact the chamber alternately.

In an embodiment, the impact modules may be arranged to face the same surface of each other based on the chamber.

In an embodiment, one impact member and another impact member of the impact modules may be configured to impact the chamber substantially simultaneously.

In an embodiment, a length of a rotation blade of one rotating member and a length of a rotation blade of another rotating member of the impact modules may be different from each other.

In an embodiment, the rotation blade may be formed of an elastic material, one end portion of the rotation blade may be a fixed end that is connected to the rotating member, and the other end portion opposite to the one end portion of the rotation blade may be a free end.

In an embodiment, the other end portion of the rotation blade may be tilted in a direction away from the chamber when the rotation blade is pressed by the impact member and the other end portion of the rotation blade may move toward the chamber and be configured to impact the chamber when the rotation blade is separated from the impact member.

In an embodiment, the inhaler may further include a puff sensor configured to sense airflow inside the stick and a processor configured to receive a sensing result from the puff sensor and control rotation of the rotating member.

### Effects

An inhaler according to an embodiment may control the amount of discharge and/or rate of discharged powder by impacting a chamber through an impact member and assist a user to smoothly inhale the powder.

The effects of the inhaler are not limited to the above-mentioned effects, and other unmentioned effects can be clearly understood from the following description by one of ordinary skill in the art to which the present disclosure pertains.

### BRIEF DESCRIPTION OF DRAWINGS

FIG. 1 is a block diagram illustrating an inhaler according to an embodiment.

FIG. 2 is a diagram schematically illustrating an inhaler according to an embodiment.

FIG. 3a is a diagram schematically illustrating the inside of an inhaler according to an embodiment.

FIG. 3b is a diagram schematically illustrating the inside of an inhaler according to an embodiment.

FIG. 4 is a diagram schematically illustrating a partial area of an inhaler according to an embodiment.

FIG. 5a is a diagram schematically illustrating a partial area of an inhaler according to an embodiment.

FIG. 5b is a diagram schematically illustrating a partial area of an inhaler according to an embodiment.

FIG. 6 is a diagram schematically illustrating a partial area of an inhaler according to an embodiment.

### BEST MODE FOR CARRYING OUT THE INVENTION

The terms used in various embodiments are selected from among common terms that are currently widely used, in consideration of their function in the disclosure. However, the terms may become different according to an intention of one of ordinary skill in the art, a precedent, or the advent of new technology. Also, in particular cases, the terms are discretionally selected by the applicant of the disclosure, and the meaning of those terms will be described in detail in the corresponding part of the detailed description. Therefore, the terms used in the disclosure are not merely designations of the terms, but the terms are defined based on the meaning of the terms and content throughout the disclosure.

It will be understood that when a certain part “includes” a certain component, the part does not exclude another component but may further include another component, unless the context clearly dictates otherwise. Also, terms such as “unit,” “module,” etc., as used in the specification may refer to a part for processing at least one function or operation and which may be implemented as hardware, software, or a combination of hardware and software.

As used herein, an expression such as “at least one of” that precedes listed components modifies not each of the listed components but all the listed components. For example, the expression “at least one of a, b, or c” should be construed as including a, b, c, a and b, a and c, band c, or a, b, and c.

In various embodiments, the term “puff” refers to inhalation by a user, and inhalation refers to a situation in which a user draws in an aerosol into his or her oral cavity, nasal cavity, or lungs through the mouth or nose.

In an embodiment, an inhaler may include a main body (or a holder) configured to support a cartridge (or a stick) configured to accommodate a capsule containing a composition. The cartridge may be detachably coupled to the main body. However, embodiments are not limited thereto. The cartridge may be integrally formed or assembled with the main body, and may be secured to the main body so as not to be detached by a user. The cartridge may be mounted on the main body while the capsule is accommodated therein. However, embodiments are not limited thereto. The powder or capsules containing the powder may be injected into the cartridge while the cartridge is coupled to the main body.

Hereinafter, embodiments of the disclosure will be described in detail with reference to the accompanying drawings such that one of ordinary skill in the art may easily practice the disclosure. The disclosure may be practiced in forms that are implementable in the inhaler according to various embodiments described above or may be embodied and practiced in many different forms and is not limited to the embodiments described herein.

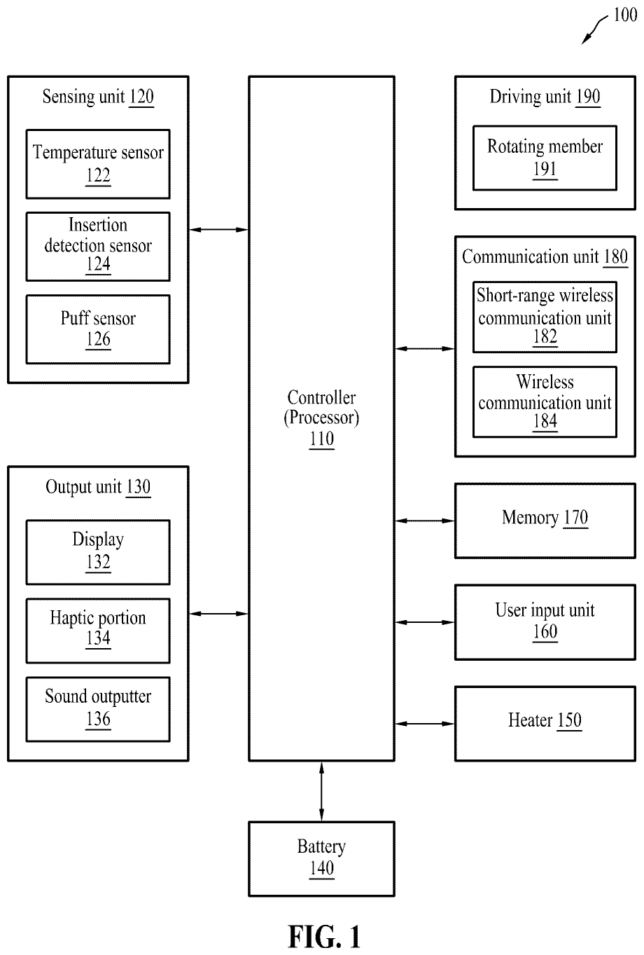

FIG. 1 is a block diagram illustrating an inhaler 100 according to an embodiment.

Referring to FIG. 1, the inhaler 100 according to an embodiment may include at least a portion of a controller 110, a sensing unit 120, an output unit 130, a battery 140, a heater 150, a user input unit 160, a memory 170, a communication unit 180, and a driving unit 190.

However, the internal structure of the inhaler 100 is not limited to that as shown in FIG. 1. It is to be understood by one of ordinary skill in the art to which the present disclosure pertains that some of the components shown in FIG. 1 may be omitted or new components may be added thereto according to the design of the inhaler 100.

In an embodiment, the sensing unit 120 may sense a state of the inhaler 100 or a state around the inhaler 100 and transmit sensed information to the controller 110 (or a processor). The controller 110 may control driving of other components of the inhaler 100 based on the sensed information.

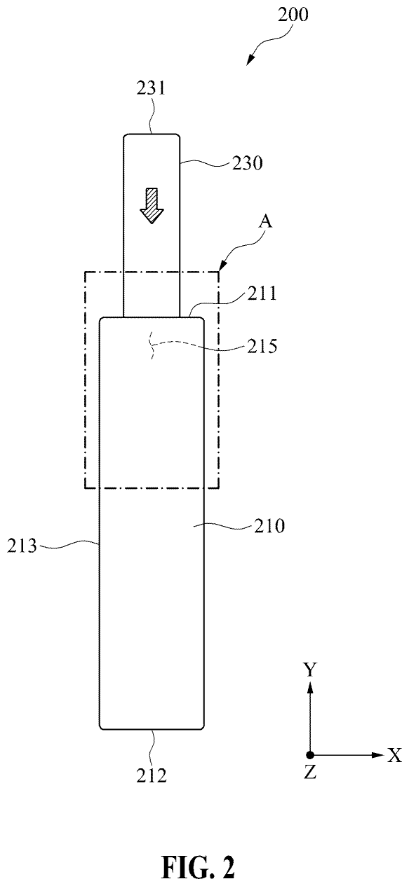

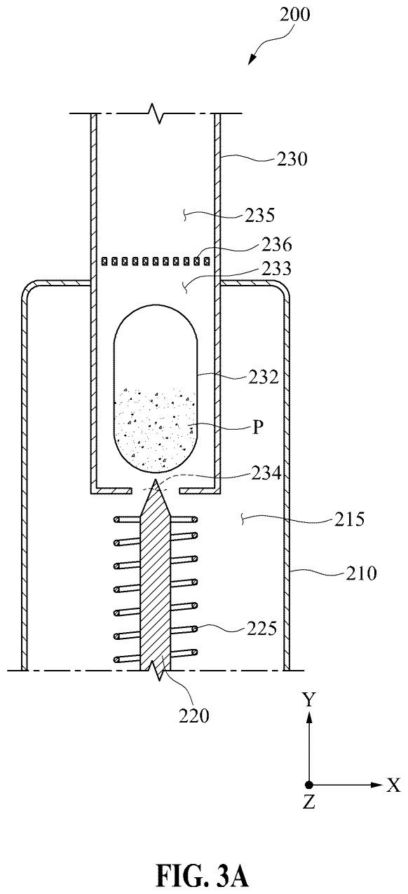

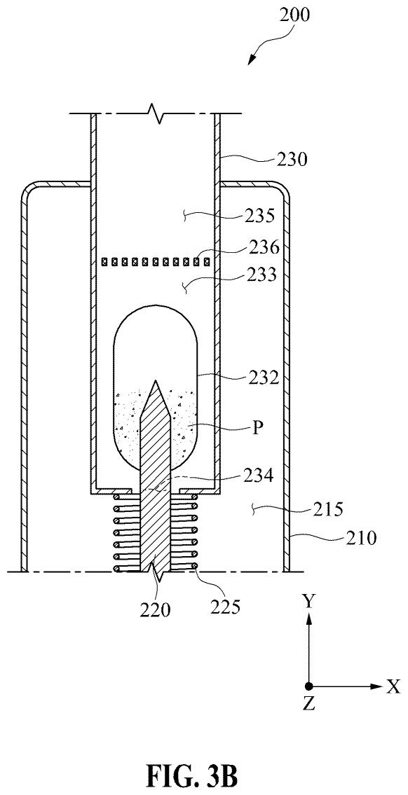

For example, the controller 110 may control an operation of the heater 150 based on a sensing result of the sensing unit 120, control the driving unit 190 by determining whether a stick (e.g., a stick 230 of FIG. 2), a capsule (e.g., a capsule 232 of FIG. 3), a cartridge, or a cigarette is inserted into an insertion groove, or perform various functions such as displaying a notification by the output unit 130.

In an embodiment, the sensing unit 120 may include at least one of a temperature sensor 122, an insertion detection sensor 124, or a puff sensor 126. However, embodiments are not limited thereto.

In an embodiment, the temperature sensor 122 may sense a temperature at which the heater 150 is heated. The inhaler 100 may include a separate temperature sensor for sensing the temperature of the heater 150, or the heater 150 itself may perform a function as a temperature sensor. Alternatively, the temperature sensor 122 may be arranged around the battery 140 to monitor the temperature of the battery 140.

In an embodiment, the insertion detection sensor 124 may detect insertion and/or removal of a stick (e.g., the stick 230 of FIG. 2) or a capsule (e.g., the capsule 232 of FIGS. 3a and 3b). The insertion detection sensor 124 may include, for example, at least one of a film sensor, a pressure sensor, a light sensor, a resistive sensor, a capacitive sensor, an inductive sensor, or an infrared sensor and may sense a signal change by the insertion and/or removal of the stick or capsule.

In an embodiment, the puff sensor 126 may sense a puff from a user based on various physical changes in an airflow path or airflow channel. For example, the puff sensor 126 may sense the puff from the user based on one of a temperature change, a flow change, a voltage change, and a pressure change.

In an embodiment, the sensing unit 120 may further include at least one of a temperature/humidity sensor, an atmospheric pressure sensor, a magnetic sensor, an acceleration sensor, a gyroscope sensor, a position sensor (e.g., a global positioning system (GPS)), a proximity sensor, or a red, green, blue (RGB) sensor (e.g., an illuminance sensor), in addition to the sensors described above. A function of each sensor may be intuitively inferable from its name by one of ordinary skill in the art, and thus, a detailed description thereof is omitted herein.

In an embodiment, the output unit 130 may output information about a state of the inhaler 100 and provide the information to the user. The output unit 130 may include at least one of a display 132, a haptic portion 134, or a sound outputter 136. However, embodiments are not limited thereto. When the display 132 and a touchpad are provided in a layered structure to form a touchscreen, the display 132 may be used as an input device in addition to an output device.

In an embodiment, the display 132 may visually provide information about the inhaler 100 to the user. For example, the information about the inhaler 100 may include at least a portion of various pieces of information such as a charging/discharging state of the battery 140 of the inhaler 100, a preheating state of the heater 150, an insertion/removal state of a stick or capsule, or a state in which the use of the inhaler 100 is restricted (e.g., detection of an abnormal item), or a vibration state of the driving unit 190, and the display 132 may externally output this information. The display 132 may be, for example, a liquid-crystal display (LCD) panel, an organic light-emitting display (OLED) panel, and the like. The display 132 may also be in the form of a light-emitting diode (LED) device.

In an embodiment, the haptic portion 134 may provide information about the inhaler 100 to the user in a haptic way by converting an electrical signal into a mechanical stimulus or an electrical stimulus. The haptic portion 134 may include, for example, a motor, a piezoelectric element, or an electrical stimulation device.

In an embodiment, the sound outputter 136 may provide the information about the inhaler 100 to the user in an auditory way. For example, the sound outputter 136 may convert an electrical signal into a sound signal and externally output the sound signal.

In an embodiment, the battery 140 may supply power to be used to operate the inhaler 100. The battery 140 may supply power to heat the heater 150.

In an embodiment, the battery 140 may supply power required for operations of the other components (e.g., the sensing unit 120, the output unit 130, the user input unit 160, the memory 170, the communication unit 180, or the driving unit 190) provided in the inhaler 100. The battery 140 may be a rechargeable battery or a disposable battery. The battery 140 may be, for example, a lithium polymer (LiPoly) battery. However, embodiments are not limited thereto.

In an embodiment, the heater 150 may receive power from the battery 140 to heat an aerosol generating material. Although not shown in FIG. 1, the inhaler 100 may further include a power conversion circuit (e.g., a direct current (DC)-to-DC (DC/DC) converter) that converts power of the battery 140 and supplies the power to the heater 150. In addition, when the inhaler 100 generates an aerosol in an induction heating manner, the inhaler 100 may further include a DC-to-alternating current (AC) (DC/AC) converter that converts DC power of the battery 140 into AC power.

In an embodiment, the controller 110, the sensing unit 120, the output unit 130, the user input unit 160, the memory 170, the communication unit 180, and the driving unit 190 may receive power from the battery 140 to perform functions. Although not shown in FIG. 1, the inhaler 100 may further include a power conversion circuit, for example, a low dropout (LDO) circuit or a voltage regulator circuit, which converts power of the battery 140 and supplies the power to respective components.

In an embodiment, the heater 150 may be formed of any suitable electrically resistive material. The suitable electrically resistive material may be, for example, a metal or a metal alloy including titanium, zirconium, tantalum, platinum, nickel, cobalt, chromium, hafnium, niobium, molybdenum, tungsten, tin, gallium, manganese, iron, copper, stainless steel, nichrome, or the like. However, embodiments are not limited thereto. In addition, the heater 150 may be implemented as a metal heating wire, a metal heating plate on which an electrically conductive track is arranged, a ceramic heating element, or the like. However, embodiments are not limited thereto.

In an embodiment, the heater 150 may be an induction heater. For example, the heater 150 may include a susceptor that heats the aerosol generating material by generating heat through a magnetic field applied by a coil.

In an embodiment, the heater 150 may include a plurality of heaters. For example, the heater 150 may include a first heater for heating an aerosol generating article and a second heater for heating a liquid.

In an embodiment, the user input unit 160 may receive information input from the user or may output information to the user. For example, the user input unit 160 may include a keypad, a dome switch, a touchpad (e.g., a contact capacitive type, a pressure resistive film type, an infrared sensing type, a surface ultrasonic conduction type, an integral tension measurement type, a piezo effect method, etc.), a jog wheel, a jog switch, or the like. However, embodiments are not limited thereto. In addition, although not shown in FIG. 1, the inhaler 100 may further include a connection interface, such as a universal serial bus (USB) interface, and may be connected to another external device through the connection interface such as a USB interface to transmit and receive information or to charge the battery 140.

In an embodiment, the memory 170, which is hardware for storing various pieces of data processed in the inhaler 100, may store data processed by the controller 110 and data to be processed thereby. The memory 170 may include at least one type of storage medium of a flash memory type memory, a hard disk type memory, a multimedia card micro type memory, a card type memory (e.g., an SD or xD memory), random access memory (RAM), static random access memory (SRAM), read-only memory (ROM), electrically erasable programmable read-only memory (EEPROM), programmable read-only memory (PROM), a magnetic memory, a magnetic disk, or an optical disk.

In an embodiment, the memory 170 may store an operating time of the inhaler 100, the maximum number of puffs, the current number of puffs, at least one temperature profile, data associated with a smoking pattern of a user, or the like.

In an embodiment, the communication unit 180 may include at least one component for communicating with another electronic device. For example, the communication unit 180 may include a short-range wireless communication unit 182 and a wireless communication unit 184.

In an embodiment, the short-range wireless communication unit 182 may include a Bluetooth communication unit, a Bluetooth Low Energy (BLE) communication unit, a near field communication unit, a wireless local area network (WLAN) communication unit (e.g., Wi-Fi), a ZigBee communication unit, an infrared data association (IrDA) communication unit, a Wi-Fi Direct (WFD) communication unit, an ultra-wideband (UWB) communication unit, and an Ant+ communication unit. However, embodiments are not limited thereto.

In an embodiment, the wireless communication unit 184 may include a cellular network communicator, an Internet communicator, a computer network (e.g., a local area network (LAN) or a wide-area network (WAN)) communicator, or the like. However, embodiments are not limited thereto. The wireless communication unit 184 may use subscriber information (e.g., international mobile subscriber identity (IMSI)) to identify and authenticate the inhaler 100 in a communication network.

In an embodiment, the driving unit 190 may include various driving devices to assist an inhalation operation of the inhaler 100 of the user. For example, the driving unit 190 may include a rotating member 191 and may assist the transfer of powder of the inhaler 100 by the rotational motion of the rotating member 191. However, embodiments are not limited thereto, and the driving unit 190 may further include elements such as a motor, a shaft, a plurality of pinions, or a hydraulic device.

In an embodiment, the rotating member 191 may be implemented as a wheel that rotates around a fixed axis, and when a voltage is applied to a power source (e.g., a motor) that rotates the rotating member 191, the rotating member 191 may rotate.

In an embodiment, the controller 110 may control the overall operation of the inhaler 100. In an embodiment, the controller 110 may include at least one processor. The processor may be implemented as an array of a plurality of logic gates, or may be implemented as a combination of a general-purpose microprocessor and a memory in which a program executable by the microprocessor is stored. In addition, it is to be understood by one of ordinary skill in the art to which the present disclosure pertains that it may be implemented in other types of hardware.

In an embodiment, the controller 110 may control the temperature of the heater 150 by controlling the supply of power from the battery 140 to the heater 150. For example, the controller 110 may control the supply of power by controlling switching of a switching element between the battery 140 and the heater 150. In another example, a direct heating circuit may control the supply of power to the heater 150 according to a control command from the controller 110.

In an embodiment, the controller 110 may analyze a sensing result obtained by the sensing of the sensing unit 120 and control processes to be performed thereafter. For example, the controller 110 may control power supplied to the heater 150 so that an operation of the heater 150 or the driving unit 190 starts or ends based on the sensing result obtained by the sensing of the sensing unit 120.

For example, the controller 110 may control the amount of power to be supplied to the heater 150 and the time for which the power is to be supplied, such that the heater 150 may be heated up to a predetermined temperature or maintained at an appropriate temperature, based on the sensing result obtained by the sensing of the sensing unit 120.

In an embodiment, the controller 110 may control the output unit 130 based on the sensing result obtained by the sensing of the sensing unit 120. For example, when the number of puffs counted through the puff sensor 126 reaches a preset number, the controller 110 may inform the user that the inhaler 100 is to end use thereof soon through at least one of the display 132, the haptic portion 134, or the sound outputter 136. Alternatively, for example, the puff sensor 126 may sense an inhalation state of the user, and based on this, the controller 110 may control driving of the rotating member 191 of the driving unit 190.

In an embodiment, the controller 110 may control the power supply time and/or the power supply amount for the heater 150 according to a state of a stick or a capsule sensed by the sensing unit 120.

An embodiment may also be implemented in the form of a recording medium including instructions executable by a computer, such as a program module executable by the computer. A computer-readable medium may be any available medium that can be accessed by a computer and includes a volatile medium, a non-volatile medium, a removable medium, and a non-removable medium. In addition, the computer-readable medium may include both a computer storage medium and a communication medium. The computer storage medium includes all of a volatile medium, a non-volatile medium, a removable medium, and a non-removable medium implemented by any method or technology for storage of information such as computer-readable instructions, data structures, program modules or other data. The communication medium typically includes a computer-readable command, a data structure, or other data regarding a modulated data signal such as a program module, or other transmission mechanisms, and includes an arbitrary information transfer medium.

FIG. 2 is a diagram schematically illustrating an inhaler 200 according to an embodiment.

Referring to FIG. 2, the inhaler 200 according to an embodiment may include at least a portion of a holder 210 and the stick 230.

In an embodiment, the holder 210 may be configured in a cylindrical or polygonal column shape. An insertion groove 215 for inserting the stick 230 may be formed in the holder 210, and the stick 230 may be inserted into the insertion groove 215 in an insertion direction (e.g., the −Y direction).

In an embodiment, the holder 210 may include a first surface 211, a second surface 212, and a side surface 213. The insertion groove 215 may be formed in the first surface 211 and the second surface 212 may be a surface opposite to the first surface 211. The side surface 213 may be formed between the first surface 211 and the second surface 212.

In an embodiment, the insertion groove 215 may be configured with a recess formed concavely in a direction from the first surface 211 toward the second surface 212 and may be formed to be opened to at least a partial area of the first surface 211.

For example, the insertion groove 215 may have a shape extending along the longitudinal axis (e.g., the +/−Y direction) of the holder 210. The stick 230 may be inserted into the holder 210 in a direction (e.g., the −Y direction) in which the insertion groove 215 penetrates.

In an embodiment, an inlet (not shown) through which air from the outside of the holder 210 may flow into the insertion groove 215 may be formed between the outside of the holder 210 and the insertion groove 215.

Although not shown in the drawing, the holder 210 may accommodate various components of the inhaler 200 therein, for example, the holder 210 may accommodate at least a portion of a controller (e.g., the controller 110 of FIG. 1), at least one sensor (e.g., the sensing unit 120 of FIG. 1), and a battery (e.g., the battery 140 of FIG. 1).

In an embodiment, the stick 230 may be configured in a cylindrical or polygonal column shape and may have a size and a shape that may be inserted into the insertion groove 215 of the holder 210. The stick 230 may accommodate powder P therein.

In an embodiment, a mouthpiece 231 may be provided on one surface of the stick 230. For example, the mouthpiece 231 may be provided in a direction opposite to an area (e.g., a chamber 233 of FIG. 3a) where the stick 230 is inserted into the insertion groove 215. The user may inhale air by applying negative pressure to the stick 230. For example, the user may inhale the powder P, or air or aerosol containing the powder P while holding the mouthpiece 231 in the mouth of the user.

FIG. 3a is a diagram schematically illustrating the inside of the inhaler 200 according to an embodiment and FIG. 3b is a diagram schematically illustrating the inside of the inhaler 200 according to an embodiment.

Specifically, FIGS. 3a and 3b are diagrams illustrating the inside of areaA shown in FIG. 2, FIG. 3a illustrates a state in which the stick 230 is partially inserted into or being inserted into the insertion groove 215 of the holder 210, and FIG. 3b illustrates a state in which the stick 230 is substantially completely inserted into the insertion groove 215 of the holder 210.

Referring to FIGS. 3a and 3b, the inhaler 200 according to an embodiment may include at least a portion of a piercing member 220, an elastic member 225, the chamber 233, and a piercing hole 234.

In an embodiment, the stick 230 may include the chamber 233 to accommodate the capsule 232. The chamber 233 may be a partial area of the stick 230 that is inserted into the insertion groove 215. The chamber 233 may be a space for accommodating or storing the capsule 232 or may be a space for limiting the movement of the capsule 232.

In an embodiment, the capsule 232 may contain the powder P therein. The powder P may be a tobacco extract in a small particle state, or the powder P may be a pharmacological material such as caffeine, taurine, aspirin, sedatives, sleeping pills, bronchodilators, or vaccines, or a composition or a functional material containing a material such as free-nicotine, or nicotine salt. However, this is only an example, and the powder P in the capsule 232 may be replaced with liquid, gas, or a combination of some of these.

In an embodiment, the piercing hole 234 may be an opening that opens toward the chamber 233 from the outside of the stick 230. The piercing hole 234 may be formed in a surface facing the insertion groove 215 from the stick 230, desirably in an area facing the piercing member 220. The piercing hole 234 may have a diameter that is greater than or equal to the circumference of the piercing member 220.

In an embodiment, the stick 230 may include an airflow channel 235 communicating from the chamber 233 to a mouthpiece (e.g., the mouthpiece 231 of FIG. 2). The airflow channel 235 may be a flow path through which air containing the powder P flows, and the airflow channel 235 and the chamber 233 may be partitioned through a mesh 236.

In an embodiment, the mesh 236 may pass the powder P and air and may limit the passage of the capsule 232 or other foreign materials. Alternatively, the mesh 236 may filter some of the powder P or help the powder P not to agglomerate. For example, the diameter of a single hole of the mesh 236 may be 5 micrometers.

In an embodiment, when the capsule 232 is crushed, at least a portion of the powder P in the capsule 232 may be discharged to the chamber 233. When the user inhales air of the stick 230 through the mouthpiece 231, the powder P may pass through the mesh 236, pass through the airflow channel 235, move to the mouthpiece 231, and be inhaled by the user.

In an embodiment, the stick 230 may be disposable and may be replaced with another stick 230 after the powder P is exhausted. Alternatively, the stick 230 may be multi-use, and when the powder P is exhausted, the stick 230 may be refilled with the capsule 232 or the powder P and used again.

In an embodiment, the piercing member 220 may be provided in the insertion groove 215 and may protrude from the insertion groove 215 in a direction (e.g., the +Y direction) facing the stick 230. The piercing member 220 may crush the capsule 232. For example, when the stick 230 is inserted into the insertion groove 215 in the first direction (e.g., the −Y direction), at least a partial area of the piercing member 220 may penetrate the piercing hole 234 and be inserted into the chamber 233 of the stick 230, and the piercing member 220 may partially crush the capsule 232.

In an embodiment, the end portion of the piercing member 220 may have a sharp or pointed shape, for example, the piercing member 220 may be a needle or a sting. The end portion of the piercing member 220 may crush a partial area of the capsule 232 and form the perforation in the capsule 232. The capsule 232 may discharge the powder P into the chamber 233 through the perforation crushed by the piercing member 220.

The elastic member 225 according to an embodiment may be provided in the insertion groove 215. When the stick 230 is inserted into the insertion groove 215, the elastic member 225 may be deformed (e.g., compressed) by being pressed by the stick 230. When the elastic member 225 is deformed, the elastic member 225 may press the stick 230 in a direction (e.g., the +Y direction) opposite to the first direction of the elastic force. The elastic member 225 may include a coil spring that may apply the elastic force to the other structure.

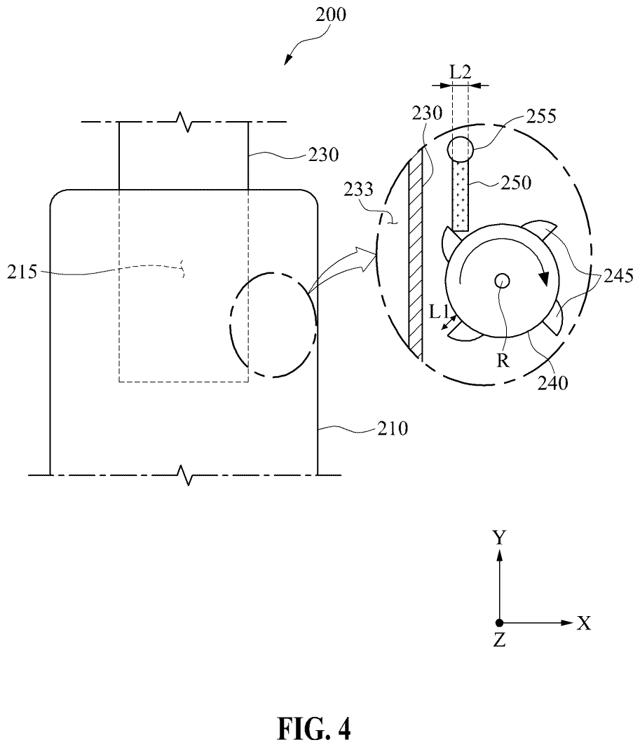

FIG. 4 is a diagram schematically illustrating a partial area of the inhaler 200 according to an embodiment.

Referring to FIG. 4, the inhaler 200 according to an embodiment may include at least one of a rotating member 240 (e.g., the rotating member 191 of FIG. 1) and an impact member 250.

In an embodiment, the rotating member 240 may be provided inside the insertion groove 215 of the holder 210. The rotating member 240 may be a rotating plate or a wheel that rotates around the rotation axis R.

In an embodiment, the rotating member 240 may receive power from a driving unit (e.g., the driving unit 190 of FIG. 1) such as an electric motor, and a rotation direction and a revolution per minute (RPM) of the rotating member 240 may be controlled by a processor (e.g., the controller 110 of FIG. 1).

In an embodiment, the rotating member 240 may include at least one rotation blade 245 provided on the outer circumferential surface of the rotating member 240. The rotation blade 245 may be formed in a plurality, and the plurality of rotation blades 245 may be spaced apart, preferably at substantially equal intervals from each other.

In an embodiment, the impact member 250 may be connected to a fixing member 255 fixed to the fixed position of the insertion groove 215 and at least a partial area of the impact member 250 may be fixed to the insertion groove 215. The fixing member 255, the impact member 250, and the rotating member 240 may be spaced apart from the outer circumferential surface of the chamber 233 by a predetermined distance and may be arranged adjacent to the chamber 233.

In an embodiment, one end portion of the impact member 250 may have a structure connected to the fixing member 255 and the other end portion of the impact member 250 may have a structure extending from the one end portion toward the rotating member 240, for example, may have a bar or cylindrical shape substantially. For example, as the rotating member 240 rotates, the other end portion of the impact member 250 may extend to a moving range of the rotation blade 245 of the rotating member 240 so that the impact member 250 and the rotation blade 245 are engaged with each other.

In an embodiment, as the rotating member 240 rotates around the rotation axis R, the rotation blade 245 of the rotating member 240 may be in contact with the impact member 250 temporarily. As the rotating member 240 continues to rotate in a situation in which the rotation blade 245 and the impact member 250 are in contact with each other, the rotation blade 245 and the impact member 250 may be engaged with each other and the rotating member 240 may temporarily press the impact member 250.

In an embodiment, the impact member 250 may be tilted by interworking with the rotation of the rotating member 240 and may impact the chamber 233. As the rotating member 240 rotates, the chamber 233 repeatedly impacted may vibrate.

Hereinafter, as the rotating member 240 rotates in one direction (e.g., clockwise of FIG. 4), an operation in which the impact member 250 formed of an elastic material impacts the chamber 233 is described in detail.

In an embodiment, the impact member 250 may be formed of an elastic material. One end portion of the impact member 250 may be a fixed end that is connected to the fixing member 255 to limit movement and the other end portion opposite to the one end portion may be a free end movable by external pressure. The impact member 250 may be temporarily deformed while being engaged with the rotation blade 245 and may impact the chamber 233 in the process of being restored.

For example, when the other end portion of the impact member 250, formed of a free end, is pressed by the rotation blade 245, the impact member 250 may be bent in a direction of the pressing. When the rotating member 240 continues to rotate and the rotation blade 245 is separated from the other end portion of the impact member 250, the impact member 250 may be bounced out by the restoring force (or the elastic force) to a position in contact with the chamber 233 beyond the position before being pressed by the rotation blade 245. The other end portion of the impact member 250 may impact the chamber 233 and the chamber 233 may vibrate due to the impact.

However, the above-described process is only an example description of a portion of the inhaler 200 according to various embodiments of the present disclosure and is not limited thereto, and the inhaler 200 may vibrate the chamber 233 through a different structure and operation.

For example, the rotating member 240 may rotate in a direction (e.g., counterclockwise) that is different from the above-described example and the rotation blade 245 may press the impact member 250 in a direction of the chamber 233. The impact member 250 may be pressed by the rotation blade 245, bounce out in the direction of the chamber 233, and impact the chamber 233. The impact member 250 may be formed of a rigid material and may be relatively freely and rotatably arranged in the insertion groove 215 with one end portion of the impact member 250 fixed to the fixing member 255.

In an embodiment, the impact member 250 may be arranged in the insertion groove 215 in a direction facing the chamber 233 and may impact a side surface of the chamber 233. For example, as shown in FIG. 4, the impact member 250 may be arranged to face one side surface of the chamber 233 (e.g., the +X direction surface) adjacent to a space into which the stick 230 is inserted.

In an embodiment, when the impact member 250 impacts the side surface of the chamber 233, the chamber 233 may vibrate in left and right directions (e.g., the X-Z plane direction). The vibration of the chamber 233 may provide vibration to a capsule (e.g., the capsule 232 of FIGS. 3a and 3b) inside the chamber 233, and as the capsule 232 vibrates, the discharge of powder (e.g., the powder P of FIGS. 3a and 3b) may be promoted. Alternatively, the vibration of the chamber 233 may provide kinetic energy to the powder P remaining in the chamber 233 and assist the powder P to move to the airflow channel 235.

However, embodiments are not limited thereto, and the impact member 250 may be arranged in the insertion groove 215 in a direction facing the chamber 233 and may impact the bottom surface of the chamber 233. For example, although not shown in the drawings, the impact member 250 may be arranged to face one surface of the stick 230 facing a direction (e.g., the −Y direction) into which the stick 230 is inserted.

In an embodiment, when the impact member 250 impacts the bottom surface of the chamber 233, the powder P remaining on the bottom surface of the chamber 233 may receive kinetic energy and rise along the airflow. Alternatively, when the impact member 250 periodically impacts the bottom surface of the chamber 233, the chamber 233 may repeatedly vibrate, provide vibration to the capsule 232 in the chamber 233, and promote the discharge of the powder P.

In an embodiment, the rotation blade 245 may have a shape protruding in a direction away from the rotation axis R. For example, one surface of the rotation blade 245 may extend in a straight line in a direction away from the rotation axis R and the other surface of the rotation blade 245 may extend in a curved shape such that the one surface and an end portion of the rotation blade 245 contact each other. However, this is only an example, and the rotation blade 245 may have various shapes such as a triangle, a quadrangle, a hemisphere, or a horn shape.

In an embodiment, the rotation blade 245 may have a first length L1 that is the width of the end portion of the rotation blade 245 in a direction away from the rotation axis R. As the first length L1 of the rotation blade 245 increases in a certain range, the compressive force applied to the impact member 250 by the rotation blade 245 may increase and the intensity of impact applied to the chamber 233 may increase.

For example, when the first length L1 of the rotation blade 245 is relatively long, the impact member 250 may be tilted at a larger angle, and when the rotation blade 245 and the impact member 250 are separated from each other, the impact member 250 may hit the chamber 233 with greater restoring force.

In an embodiment, the impact member 250 may have a second length L2 that is a width in a tilted direction (e.g., the X-axis direction). As the second length L2 of the impact member 250 increases in a certain range, the compressive force stored by the impact member 250 may increase and the intensity of impact applied to the chamber 233 may increase.

For example, when the second length L2 of the impact member 250 is relatively long, the rotation blade 245 must press the impact member 250 with a large pressure for tilting the impact member 250, and when the rotation blade 245 and the impact member 250 are separated from each other, the impact member 250 may hit the chamber 233 with greater restoring force.

In an embodiment, the inhaler 200 may need to reduce or increase the amount or rate of discharge of the powder P from the capsule 232 according to various factors such as the respiration volume of the user or the preference of the user.

In various embodiments of the present disclosure, the impact member 250 may impact the chamber 233 by interworking with the rotation of the rotating member 240. The chamber 233 may control the powder P to be efficiently discharged from the capsule 232 by vibrating by the impact and may assist the powder P to move to the airflow channel 235 efficiently.

For example, the processor 110 may receive a sensing result from a puff sensor (e.g., the puff sensor 126 of FIG. 1) that senses the airflow inside the stick 230 and based on this, may control the rotation direction of the rotating member 240 and a rotation element such as the RPM. For example, when it is determined that the lung capacity of the user is relatively low, the processor 110 may increase the RPM of the rotating member 240 and may increase the intensity of the vibration and/or the number of vibrations applied to the chamber 233.

As described above, the rotating member 240 and the impact member 250 according to various embodiments of the present disclosure may be arranged in various positions and may have various shapes and sizes, and based on this, factors such as the intensity, direction, and cycle of the impact applied to the chamber 233 by the impact member 250 may be controlled.

In an embodiment, the fixing member 255, the impact member 250, and the rotating member 240 may configure one impact module. The impact modules may refer to one set including one impact member 250 capable of impacting a preset impact point of the chamber 233 and the rotating member 240 for implementing the impact of the impact member 250. Hereinafter, the inhaler 200 including the plurality of impact modules is described with reference to the above description.

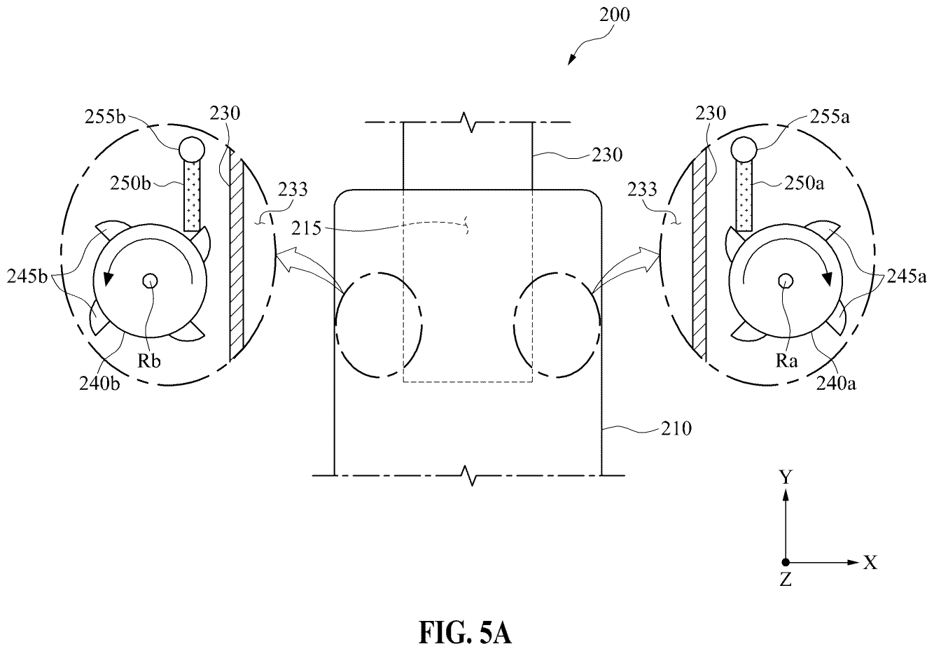

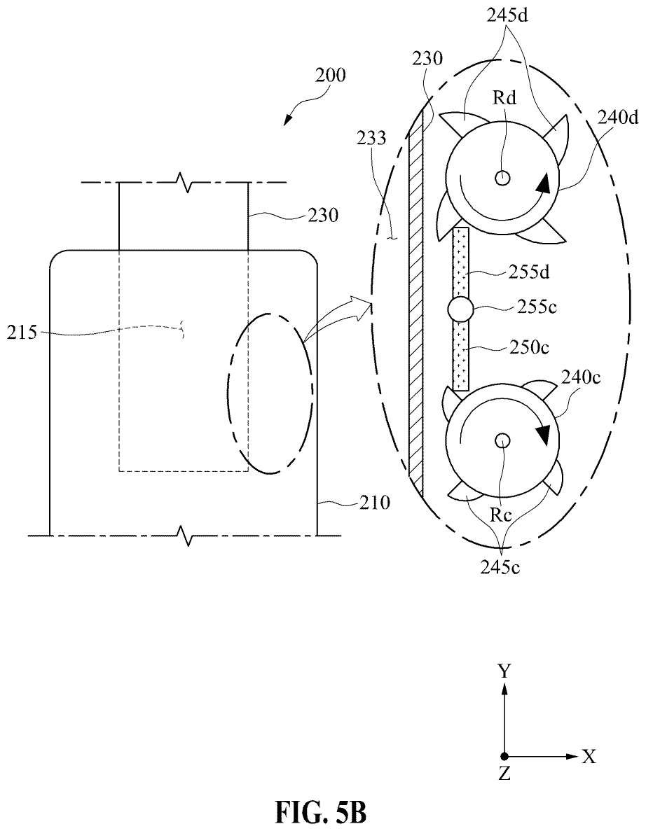

FIG. 5a is a diagram schematically illustrating a partial area of the inhaler 200 according to an embodiment.

Referring to FIG. 5a, the inhaler 200 may include a plurality of impact modules.

Hereinafter, the inhaler 200 that impacts areas of the chamber 233 including a plurality of rotating members 240a and 240b and a plurality of impact members 250a and 250b is described and a repeated description related thereto is omitted.

In an embodiment, the plurality of impact modules may include first impact modules and second impact modules. The first impact modules may include a first impact member 250a and a first rotating member 240a, and the second impact modules may include a second impact member 250b and a second rotating member 240b.

In an embodiment, the first impact modules and the second impact modules may be arranged in opposite directions (e.g., the +/−X directions) with each other based on the chamber 233.

For example, the first rotating member 240a and the second rotating member 240b may rotate in opposite directions to each other based on a first rotation axis Ra and a second rotation axis Rb, respectively. As the first impact member 250a is pressed by a first rotation blade 245a of the first rotating member 240a, in a state in which one end portion of the first impact member 250a is fixed to a first fixing member 255a, the other end portion of the first impact member 250a may be tilted in a direction away from the chamber 233. As the second impact member 250b is pressed by a second rotation blade 245b of the second rotating member 240b, in a state in which one end portion of the second impact member 250b is fixed to a second fixing member 255b, the other end portion of the second impact member 250b may be tilted in a direction away from the chamber 233. In addition, the first impact member 250a and the second impact member 250b may respectively impact different areas (e.g., facing areas) of the chamber 233 by the restoring force. For example, the first impact member 250a may impact one side (e.g., the +X side) of the side surface of the chamber 233 and the second impact member 250b may impact the other side (e.g., the −X side) of the side surface of the chamber 233.

In an embodiment, the first impact member 250a and the second impact member 250b may repeatedly impact the chamber 233 in both directions of the chamber 233, respectively, so that the chamber 233 may receive stronger vibration and may transmit the stronger vibration to a capsule (e.g., the capsule 232 of FIGS. 3a and 3b) and powder (e.g., the powder P of FIGS. 3a and 3b). Alternatively, the first impact member 250a and the second impact member 250b may supply kinetic energy to the powder P in both directions, so that the powder P may be induced to spread evenly.

In an embodiment, in the first impact modules and the second impact modules, the first impact member 250a and the second impact member 250b may impact the chamber 233 alternately. As the first impact member 250a and the second impact member 250b alternately impact the chamber 233, the intensity of a single vibration transmitted to the inhaler 200 may decrease, the magnitude of the entire vibration transmitted to the chamber 233 may increase, and the effect of the vibration may be improved.

FIG. 5b is a diagram schematically illustrating a partial area of the inhaler 200 according to an embodiment.

Referring to FIG. 5b, the inhaler 200 may include a plurality of impact modules.

Hereinafter, the inhaler 200 that impacts areas of the chamber 233 including a plurality of rotating members 240c and 240d and the plurality of impact members 250c and 255d is described and a repeated description related thereto is omitted.

In an embodiment, the plurality of impact modules may include third impact modules and fourth impact modules.

In an embodiment, the third impact modules may include a third impact member 250c and a third rotating member 240c, and the fourth impact modules may include a fourth impact member 255d and a fourth rotating member 240d.

In an embodiment, the third impact modules and the fourth impact modules may be arranged to face the same surface of the chamber 233.

In an embodiment, one end portion of the third impact member 250c of the third impact modules and one end portion of the fourth impact member 255d of the fourth impact modules may be connected to a third fixing member 255c equally. One end portion of the third impact member 250c may extend from the other end portion of the third impact member 250c that is connected to the third fixing member 255c in one direction (e.g., the +Y direction) and one end portion of the fourth impact member 255d may extend from the other end portion of the fourth impact member 255d that is connected to the third fixing member 255c in the other direction (e.g., the −Y direction).

For example, the third rotating member 240c and the fourth rotating member 240d may rotate in opposite directions to each other based on a third rotation axis Rc and a fourth rotation axis Rd, respectively. As the third impact member 250c is pressed by a third rotation blade 245c of the third rotating member 240c, in a state in which the other end portion of the third impact member 250c is fixed to the third fixing member 255c, one end portion of the third impact member 250c may be tilted in a direction away from the chamber 233. As the fourth impact member 255d is pressed by a fourth rotation blade 245d of the fourth rotating member 240d, in a state in which the other end portion of the fourth impact member 255d is fixed to the third fixing member 255c, one end portion of the fourth impact member 255d may be tilted in a direction away from the chamber 233. In addition, the third impact member 250c and the fourth impact member 255d may respectively impact different areas of one surface of the chamber 233 by the restoring force. For example, the third impact member 250c may impact an area of one side (e.g., the −Y side or lower side) of the side surface of the chamber 233 and the fourth impact member 255d may impact an area of the other side (e.g., the +Y side or upper side) of the side surface of the chamber 233.

In an embodiment, as the third impact member 250c and the fourth impact member 255d impact positions spaced apart from each other on the same surface of the chamber 233, the chamber 233 may receive stronger vibration overall and the stronger vibration may be transmitted to a capsule (e.g., the capsule 232 of FIGS. 3a and 3b) and powder (e.g., the powder P of FIGS. 3a and 3b).

However, embodiments are not limited thereto, and the third impact modules and the fourth impact modules may be independently arranged spaced apart from each other by being connected to the respective fixing members 255, and each may impact different areas of the chamber 233.

In an embodiment, the length (e.g., the first length L1 of FIG. 4) of the third rotation blade 245c and the length of the fourth rotation blade 245d may be different from each other. In response to the need for a stronger or weaker impact, the lengths of the rotation blades 245c and 245d may be set differently.

For example, when the length of the fourth rotation blade 245d is longer than the length of the third rotation blade 245c, the fourth impact member 255d may efficiently generate vibration to the chamber 233 by strongly impacting a position where the stronger vibration is required.

However, embodiments are not limited thereto, and the width (e.g., the second length L2 of FIG. 4) of the third impact member 250c and the width of the fourth impact member 255d may be different from each other, and in response to the need for a stronger or weaker impact, the lengths of the impact members 250c and 255d may be set differently. The description that the length L1 of the plurality of rotation blades 245c and 245d and/or the length L2 of the plurality of impact members 250c and 255d are different from each other may also apply to the embodiment of FIG. 5a or other embodiments.

In an embodiment, as the third impact member 250c and the fourth impact member 255d may impact the chamber 233 in the same direction of the chamber 233, respectively, the chamber 233 may receive stronger vibration and the stronger vibration may be transmitted to the capsule 232 and the powder P.

In an embodiment, in the third impact modules and the fourth impact modules, the third impact member 250c and the fourth impact member 255d may impact the chamber 233 substantially simultaneously. As the third impact member 250c and the fourth impact member 255d simultaneously impact the chamber 233, the intensity of a single vibration transmitted to the inhaler 200 may decrease, the magnitude of the entire vibration transmitted to the chamber 233 may increase and the effect of the vibration may be improved.

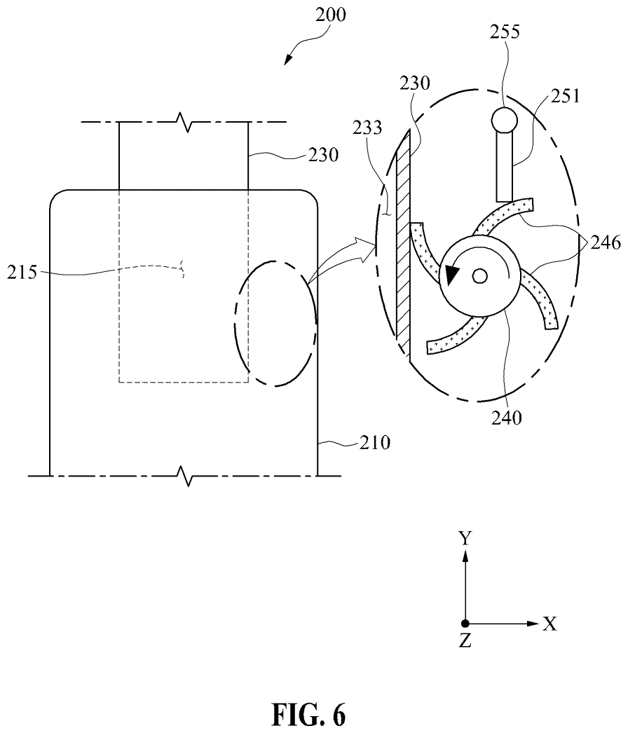

FIG. 6 is a diagram schematically illustrating a partial area of the inhaler 200 according to an embodiment.

Referring to FIG. 6, a rotation blade 246 of the rotating member 240 according to an embodiment may impact the chamber 233.

Hereinafter, the inhaler 200 in which the rotation blade 246 impacts the chamber 233 is described, and for convenience of description, a repeated description related thereto is omitted and the different descriptions are mainly provided.

In an embodiment, the rotation blade 246 may be formed of an elastic material. One end portion of the rotation blade 246 may be a fixed end that is connected to the rotating member 240 and the other end portion opposite to the one end portion may be a free end. As the rotating member 240 rotates, the rotation blade 246 may impact a sidewall of the chamber 233.

In an embodiment, the other end portion of the impact member 251 may also be a fixed end in addition to the one end portion that is connected to the fixing member 255. The rotation blade 246 may be temporarily deformed while being engaged with the impact member 251 and may impact the chamber 233 in the process of being restored.

For example, when the other end portion of the rotation blade 246, which is a free end, is pressed by the impact member 251, the rotation blade 246 may be bent in a direction of the pressing. In addition, when the rotating member 240 continues to rotate and the other end portion of the rotation blade 246 is separated from the impact member 251, the rotation blade 246 may be bounced out to a position in contact with the chamber 233 by the restoring force (or the elastic force). The other end portion of the rotation blade 246 may impact the chamber 233 and the chamber 233 may vibrate due to the impact. The impact member 251 may be separated from the rotation blade 246 after compressing the rotation blade 246, and the intensity of impact applied to the chamber 233 by the rotation blade 246 may increase.

In an embodiment, by repeatedly impacting the chamber 233 according to rotation of the rotation blade 246, the chamber 233 may receive vibration and transmit the vibration to a capsule (e.g., the capsule 232 of FIGS. 3a and 3b) and powder (e.g., the powder P of FIGS. 3a and 3b). The rotation blade 246 may supply kinetic energy to the powder P to induce the powder P to spread evenly.

While the embodiments are described with reference to drawings, it will be apparent to one of ordinary skill in the art that various alterations and modifications in form and details may be made in these embodiments without departing from the spirit and scope of the claims and their equivalents. For example, suitable results may be achieved if the described techniques are performed in a different order and/or if components in a described system, architecture, device, or circuit are combined in a different manner and/or replaced or supplemented by other components or their equivalents. Therefore, other implementations, other embodiments, and/or equivalents of the claims are within the scope of the following claims.

1. An inhaler comprising:
a stick comprising a chamber configured to accommodate powder;
a holder comprising an insertion groove into which the stick is inserted;
a fixing member fixed to the insertion groove;
an impact member connected to the fixing member; and
a rotating member comprising a rotation axis provided in the insertion groove and at least one rotation blade provided on an outer circumferential surface of the rotating member,
wherein the rotating member is configured to press the impact member temporarily while the rotation blade and the impact member are engaged with each other, as the rotating member rotates around the rotation axis.

2. The inhaler of claim 1, wherein one end portion of the impact member is a fixed end that is connected to the fixing member and the other end portion opposite to the one end portion of the impact member is a free end.

3. The inhaler of claim 2, wherein the other end portion of the impact member is tilted in a direction away from the chamber when the impact member is pressed by the rotation blade and the other end portion of the impact member moves toward the chamber and is configured to impact the chamber when the impact member is separated from the rotation blade.

4. The inhaler of claim 2, wherein the other end portion of the impact member bounces toward the chamber and is configured to impact the chamber when the impact member is pressed by the rotation blade.

5. The inhaler of claim 1, wherein the impact member is arranged to face one surface of the stick facing a direction into which the stick is inserted in the insertion groove.

6. The inhaler of claim 1, wherein the impact member is arranged to face a side surface adjacent to one surface of the stick facing a direction into which the stick is inserted in the insertion groove.

7. The inhaler of claim 1, wherein
the impact member and the rotating member configure one impact module, and
the inhaler comprises the impact module in a plurality.

8. The inhaler of claim 7, wherein the impact modules are arranged in opposite directions to each other based on the chamber.

9. The inhaler of claim 7, wherein one impact member and another impact member of the impact modules are configured to impact the chamber alternately.

10. The inhaler of claim 7, wherein the impact modules are arranged to face the same surface of the chamber.

11. The inhaler of claim 7, wherein one impact member and another impact member of the impact modules are configured to impact the chamber substantially simultaneously.

12. The inhaler of claim 7, wherein a length of a rotation blade of one rotating member and a length of a rotation blade of another rotating member of the impact modules are different from each other.

13. The inhaler of claim 1, wherein the rotation blade is formed of an elastic material, one end portion of the rotation blade is a fixed end that is connected to the rotating member, and the other end portion opposite to the one end portion of the rotation blade is a free end.

14. The inhaler of claim 13, wherein the other end portion of the rotation blade is tilted in a direction away from the chamber when the rotation blade is pressed by the impact member and the other end portion of the rotation blade moves toward the chamber and is configured to impact the chamber when the rotation blade is separated from the impact member.

15. The inhaler of claim 1, further comprising:
a puff sensor configured to sense airflow inside the stick; and
a processor configured to receive a sensing result from the puff sensor and control rotation of the rotating member.
